(19) United States
(12) Patent Application Publication
(10) Pub. No.: US 2022/0015718 A1
(43) Pub. Date: Jan. 20, 2022

(54) METHOD OF APPROXIMATING A PATIENT'S PULSE WAVE BASED ON NON-INVASIVE BLOOD PRESSURE MEASUREMENT, A LOGIC UNIT THEREFORE AND A SYSTEM THEREFORE

(71) Applicant: PHILIPS MEDIZIN SYSTEME BÖBLINGEN GMBH, BOBLINGEN (DE)

(72) Inventors: Reinhold KNOLL, NEUBURG (DE); Ulrich PFEIFFER, MUNCHEN (DE)

(21) Appl. No.: 17/474,668

(22) Filed: Sep. 14, 2021

Related U.S. Application Data
(63) Continuation of application No. 14/761,649, filed on Jul. 17, 2015, now abandoned.
(60) Provisional application No. 61/756,895, filed on Jan. 25, 2013.

(30) Foreign Application Priority Data
Jan. 25, 2013 (EP) 13000376.7

(51) Int. Cl. A61B 5/00; A61B 5/022; A61B 5/021

(52) U.S. Cl. CPC A61B 5/7278; A61B 5/02233; A61B 5/7225; A61B 5/7264; A61B 2560/0223; A61B 5/02225; A61B 5/6824; A61B 5/725; A61B 5/02108

(57) ABSTRACT

In an approach to non-invasive blood pressure measurements, a blood pressure measurement system receives a pulse curve signal representing a sequence of pulse curves of a subject from a pressure cuff. The blood pressure measurement system applies a weighting function that applies one or more weights to a pulse curve of the sequence of pulse curves to obtain a weighted pulse curve and the one or more non-constant clamp pressure signals are a weighting function input parameter. The blood pressure measurement system sums at least the weighted pulse curve and a second weighted pulse curve, which approximates a shape and an amplitude of a pulse wave of the subject. The blood pressure measurement system determines a health status of the subject based on the pulse wave and generates a graphical representation of the pulse wave of the subject and the health status of the subject.

The present invention relates to a method of approximating a patient's pulse wave based on non-invasive blood pressure measurement. The invention also relates to a logic unit and a corresponding system for approximating a patient's pulse wave based on non-invasive blood pressure measurement.

A skilled practitioner, such as an experienced physician, can obtain useful information as to the health status of a patient from an analysis of the curve progression of the arterial blood pressure, i.e. pulse wave, of the patient. The pulse wave of the patient may be reliably measured in an invasive way, by introducing a catheter into one of the patient's blood vessels. However, invasive blood pressure measurement approaches are relatively complex, sometimes being accompanied by adverse side-effects for the patient, such as thrombo-embolic complications, bleedings and infections.

A well-known, less dangerous and more convenient way to determine the arterial blood pressure values of a patient is to use the so-called “oscillometric non-invasive blood pressure measurement method”. By that method, a pressure cuff is applied to one of the patient's extremities, preferably to his upper arm at the level of his heart, as schematically illustrated in FIG. 1. Then, the pressure in the pressure cuff is increased or decreased, usually at a constant rate, thereby exerting pressure on an artery in the patient's extremity. For example, the pressure in the pressure cuff may be increased from a value equal to or smaller than the diastolic arterial blood pressure DAP to a value equal to or greater than the systolic arterial blood pressure SAP of the patient. That is, the pressure in the pressure cuff is continuously increased over a time period which corresponds to a plurality of heart beats.

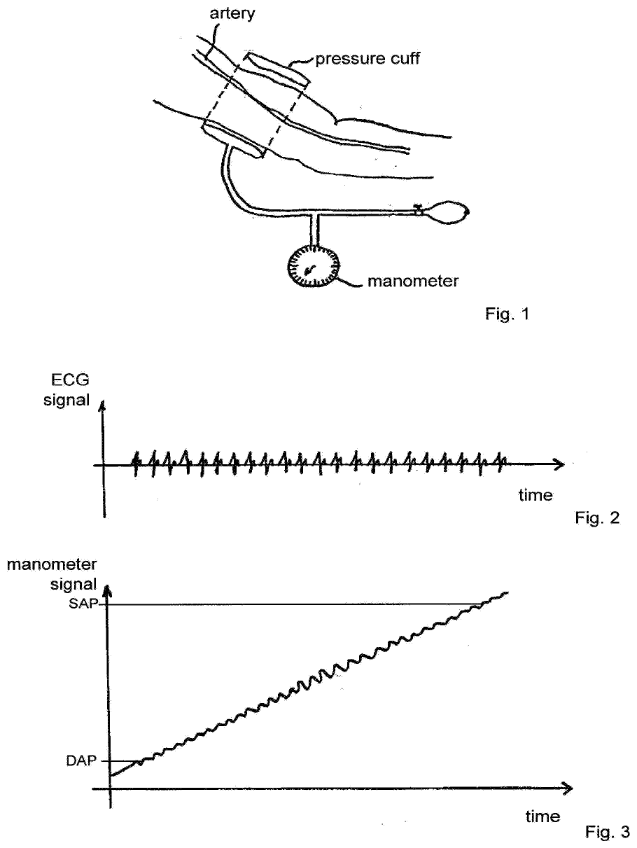

FIG. 2 schematically illustrates an electrocardiogram signal (ECG-signal) over the time. The manometer connected to the pressure cuff (shown in FIG. 1) not only indicates the continuously increasing pressure applied to the pressure cuff, but in addition (due to the principle action=reaction) also the pulses, as schematically illustrated in FIG. 3. In the following, the term “pulse” refers to pressure oscillations caused by one heart beat of the patient.

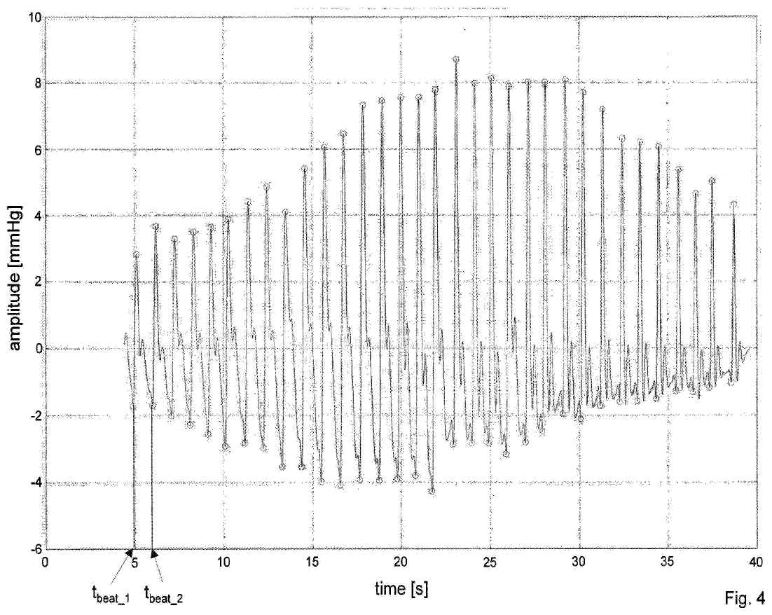

FIG. 4 is an exemplary diagram showing exclusively the pulses (i.e. pressure oscillations caused by the patient's heart beats) indicated by the manometer over the time (the pressure variation caused by the continuously increasing cuff pressure is omitted in this diagram). As shown in this diagram, a sequence of pulse signals (caused by a corresponding number of heart beats of the patient) has been measured. The pressure oscillations shown in FIG. 4 are plotted in such a way that the curve cyclically oscillates around a zero pressure line (i.e. zero pressure value). The area enclosed by the curve below the zero pressure line substantially corresponds to the area enclosed by the curve above the zero pressure line. Small circles in FIG. 4 indicate the lower and upper extreme values associated to the individual heart beats. That is, a single heart beat of the patient causes a pulse lasting from a first lower extreme value of the curve to a subsequent, second lower extreme value of the curve. This way of representation of a sequence of pulse signals measured by the so-called “oscillometric non-invasive blood pressure measurement method”, and how to determine the lower and upper extreme values associated to the individual heart beats, is well known to those skilled in the art.

The distance between two subsequent lower (or upper) extreme values of the curve shown in FIG. 4 is substantially constant (corresponding to the patient's heart rate). However, the amplitude and the general shape of the measured pulse signals associated to the individual heart beats significantly differ from each other (even though, the actual pulse waves of the patient remain substantially unchanged over the detection time). For example, the amplitude of the measured pulse signals associated to the individual heart beats is not constant, but the curve shown in FIG. 4 is rather bell-shaped. Furthermore, the pulse signals measured at higher cuff pressures are more ragged than those measured at lower cuff pressures. This phenomenon is characteristic for pulse signals measured by the so called “oscillometric non-invasive blood pressure measurement method”.

The above described oscillometric non-invasive blood pressure measurement method is relatively popular, because it enables a skilled practitioner to easily determine the systolic arterial blood pressure SAP and the diastolic arterial blood pressure DAP of a patient (by using an empirical approach). It is known that the oscillation amplitude is between 45-57%, usually 50%, of the maximum oscillation amplitude at a clamp pressure equal to the systolic arterial blood pressure SAP, whereas the oscillation amplitude is between 75-86%, usually 80%, of the maximum oscillation amplitude at a clamp pressure equal to the diastolic arterial blood pressure DAP. Thus, the absolute pressure values indicated by the manometer at corresponding moments correspond to the diastolic arterial blood pressure DAP and the systolic arterial blood pressure SAP. Notably, instead of a classic manometer, an electrical sensor may be equally applied. The above-described principle is conferrable to other physical values, such as acceleration, sound and optical reflection.

Disadvantageously, it is impossible with this known blood pressure measurement method to reliably determine the shape of the pulse wave of the patient's arterial blood pressure. As mentioned above, the exact shape of the patient's pulse wave, however, can provide important information (as to the health status of this patient) to a skilled practitioner, such as an experienced physician.

EP 0 078 090 A1 describes a non-invasive blood pressure measurement method that is—at least theoretically—capable of determining the arterial pulse wave of a patient. According to this method, a fluid-filled pressure cuff is attached to a patient's finger. A light source and a light detector are integrated in the pressure cuff, the light source and the light detector forming part of a photo-electric plethysmograph. The cuff pressure is controlled—via a fast-acting electric pressure valve—in closed-loop operation based on the plethymographic signal, so that the arterial volume in the finger is maintained at a predefined value. Measuring the pressure in the pressure cuff, thus, allows for determining the arterial blood pressure of the patient. This method is also known in literature as “volume-clamp-method”.

However, permanently correcting or re-adjusting the pressure in the pressure cuff in real time is technically difficult and prone to errors. Furthermore, till now, this method only works with a pressure cuff applied to a patient's finger. The finger is yet located relatively remote from the patient's heart, so that the diameter of arterial vessels in the finger is relatively small compared to the diameter of arterial vessels close to the heart. Due to interference effects caused by pressure reflections occurring when the diameter of arterial vessels (abruptly) changes, e.g. when arterial vessels branch, the blood pressure measurable at the finger only imprecisely corresponds to the arterial pulse wave at the patient's heart. To consider these interference effects, it was tried to reconstruct the pulse curve in the patient's aorta from the signals measured at the patient's finger, using transfer functions that are usually based on empirical and statistical parameters. However, since the parameters are not (or not sufficiently) adapted to the individual patient and situation, such an approach is not promising, potentially providing imprecise results.

It is therefore the object of the present invention to provide a method and a corresponding device for better approximating a patient's central arterial pulse wave based on non-invasive blood pressure measurement.

This object is achieved by the subject-matters of the independent claims. Preferred embodiments are the subject-matter of the dependent claims.

According to a first aspect of the present invention, there is provided a method of approximating a patient's pulse wave based on non-invasive blood pressure measurement, comprising the following steps:
(a) non-invasively measuring a sequence n=1 . . . N of pulse signals pulse_{n_measured}(t) of a patient, thereby applying a clamp pressure clampₙ(t);
(b) weighting the measured pulse signals pulse_{n_measured}(t) using a weighting function to obtain weighted pulse signals pulse_{n_weighted}(t);
(c) adding up the weighted pulse signals pulse_{n_weighted}(t) to obtain an approximation of the patient's pulse wave pulseₐₚₚᵣₒₓ(t).

A good approximation of the patient's pulse wave pulseₐₚₚᵣₒₓ(t) can be obtained simply by weighting a corresponding number of measured pulse signals pulse_{n_measured}(t) and then adding up the weighted pulse signals pulse_{n_weighted}(t). The method according to the present invention allows—without difficulty—for measuring the pulse signals close to the patient's heart, e.g. at the patient's upper arm, so as to substantially avoid interference effects otherwise occurring when measuring the pulse signals at locations remote from the patient's heart, e.g. at a finger of the patient, as with the above-described volume-clamp-method. Furthermore, the method according to the present invention allows for determining an approximation of the patient's pulse wave without the need of permanently correcting or re-adjusting the pressure in the pressure cuff, like with the volume-clamp-method.

In method step (a) of the inventive method, a sequence n=1 . . . N of pulse signals pulse_{n_measured}(t) of a patient is measured in an non-invasive fashion, using a (not-constant) clamp pressure clampₙ(t). N corresponds to the total number of individual heart beats of the patient within the detection period. In the exemplary diagram shown in FIG. 4, about 32 “pulse waves” corresponding to 32 individual heart beats have been measured within the detection period. Therefore, in this example, N=32 and, thus, n=1 . . . 32.

Preferably, the well-known and relatively comfortable “oscillometric non-invasive blood pressure measurement method” (as described above) is applied to measure the patient's pulse signals. In this method, the clamp pressure clampₙ(t) is applied to an extremity of the patient, preferably to an upper arm of the patient, as shown e.g. in FIG. 1. The clamp pressure (clampₙ(t)) is preferably varied between a value equal to or smaller than the diastolic arterial blood pressure DAP and a value equal to or greater than the systolic arterial blood pressure SAP of the patient. As known in the art, for this purpose SAP and DAP is estimated or derived from previous measurements.

Thereby, the clamp pressure clampₙ(t) may be increased or decreased continuously, preferably at a substantially constant rate. Notably, the increase or decrease rate should be low enough so as to detect a sufficient number of pulses caused by individual heart beats of the patient (preferably at least 10). The clamp pressure clampₙ(t) may be continuously increased or decreased between the diastolic arterial blood pressure DAP and the systolic arterial blood pressure SAP within a detection period of e.g. about one minute. During this detection period, pulse signals pulse_{n_measured}(t) associated to e.g. 60 individual heart beats of the patient may be measured, which represents a very good base for the further method steps. However, to avoid problems caused by blockage of blood circulation in the patient's extremity to which the pressure cuff is applied, the increase or decrease rate of the clamp pressure clampₙ(t) should not be too low, i.e. the detection time should preferably not exceed one minute.

If the increase or decrease rate of the clamp pressure clampₙ(t) is moderate (e.g. the detection time is about one minute), the clamp pressure clampₙ(t) associated with the time period of one individual heart beat might be considered—for the sake of simplicity—as being substantially constant. For example, the clamp pressure clampₙ(t) associated with the time period of one individual heart beat might be approximated so as to correspond to the actual clamp pressure at the beginning (t=t_{beat_n}) of the corresponding heart beat (clampₙ=clamp(t_{beat_n})). In the example shown in FIG. 4, the clamp pressure clampₙ(t) associated with the first detected heart beat (n=1) thus corresponds to the clamp pressure at the time beginning of the first heart beat at t=t_{beat_1}=5s, i.e. clamp₁=clamp(t_{beat_1}). Accordingly, the clamp pressure clampₙ(t) associated with the second detected heart beat (n=2) corresponds to the clamp pressure at the beginning of the second heart beat at t=t_{beat_2}=6s, i.e. clamp₂=clamp(t_{beat_2}).

However, the clamp pressure clampₙ(t) associated with the time period of one individual heart beat might equally be approximated so as to correspond e.g. to the actual clamp pressure at the end or somewhere in the middle (preferably exactly in the middle) of the corresponding heart beat.

Notably, if the sequence of pulse signals pulse_{n_measured}(t) has been previously measured and stored, the method step (a) might be skipped and the method according to the present invention may directly start with method step (b) based on the previously stored signal values.

In method step (b) of the inventive method, the measured pulse signals pulse_{n_measured}(t) are weighted to obtain weighted pulse signals pulse_{n_weighted}(t). A weighting function is applied in method step (b), as will be described in more detail below.

Finally, in method step (c), the weighted pulse signals pulse_{n_weighted}(t) are added up to obtain the approximation of the patient's pulse wave pulseₐₚₚᵣₒₓ(t). In method step (c), the approximation of the patient's pulse wave pulseₐₚₚᵣₒₓ(t) might be simply calculated as follows:

$${pulse}_{approx} ( t ) = {\sum }_{n = 1}^{N} {pulse}_{n \_ weighted} ( t ) .$$

As set forth above, the measured pulse signals pulse_{n_measured}(t), i.e. the cyclic pressure variations corresponding to the individual heart beats, detected by the manometer (as shown in FIG. 4), not only vary with respect to their amplitude, but they are also significantly distorted as to the shape of the pulse waves. Even though, this phenomenon is not yet completely understood, it is supposed that it is mainly caused by the non-resilience of the body tissue between the artery and the pressure cuff.

The inventors have found out that at moments, when the actual internal pressure (i.e. arterial blood pressure) equals a predetermined difference, e.g. approximately zero, to the externally applied pressure (i.e. cuff pressure), there exists a substantially linear relationship between the measured pulse signals and the actual arterial blood pressure (for example, at moments when the applied cuff pressure substantially equals the actual internal arterial blood pressure, the body tissue between the artery, e.g. in the upper arm, and the pressure cuff is relaxed, i.e. not biased).

Therefore, it is advantageous—in order to obtain an improved approximation of the patient's pulse wave—to use the clamp pressure clampₙ(t) as an input parameter of the weighting function, wherein the weighting function is preferably a differential pressure function. That is, the measured pulse signals pulse_{n_measured}(t) may be weighted in such a way that those portions of the curve of the measured pulse signals pulse_{n_measured}(t) are more “emphasised” that have been measured during moments at which the actual internal arterial blood pressure equals a predetermined percentage of the externally applied cuff pressure.

However, a problem resides in that usually the moments, at which the actual internal arterial blood pressure equals a predetermined percentage of the externally applied cuff pressure, are unknown, because the actual internal arterial blood pressure (i.e. the patient's pulse wave) is unknown. In fact, an approximation of the patient's pulse wave is the pursued result of the method.

To overcome this problem, method steps (b) and (c) of the method according to the present invention are preferably iteratively repeated at least one more time. The outcome of the first iteration loop, i.e. the approximation of the patient's pulse wave, can then be used as approximation of the actual internal arterial blood pressure in the second iteration loop. Thus, the moments, at which the actual internal arterial blood pressure equals a predetermined percentage of the externally applied cuff pressure, can be (at least approximately) determined. In the second iteration loop, the measured pulse signals pulse_{n_measured}(t) can then be weighted accordingly (in step (b) of the second iteration loop) before adding up the weighted pulse signals pulse_{n_weighted}(t) (in step (c) of the second iteration loop) so as to obtain an improved approximation of the patient's pulse wave pulseₐₚₚᵣₒₓ(t).

The outcome of the method can be even further improved by iteratively repeating method steps (b) and (c) more than two times, wherein the outcome of method step (c) of the previously iteration loop is used as input value for the present iteration loop. Preferably, the weighting function of the present iteration loop is a differential pressure function comprising, as an input parameter, the externally applied cuff pressure and, as another input parameter, the approximation of the actual internal arterial blood pressure, i.e. the approximated patient's pulse wave pulseₐₚₚᵣₒₓ(t) determined in the previous iteration loop.

Of course, it is not possible to determine the weighting function to be applied in the very first iteration loop that way, since there is no result of a previous iteration loop available as input. Consequently, the weighting function applied in the first iteration loop preferably differs from the weighting function applied in the second and/or higher iteration loop. The first iteration loop, thus, provides a more roughly approximated pulse wave pulseₐₚₚᵣₒₓ(t) of the patient compared to following iteration loops. For example, the weighting function of the first iteration loop might simply be determined as follows:

weight1ₙ=1 if DAP<clampₙ(t)<SAP,

weight1ₙ=0 otherwise,

wherein DAP corresponds to the diastolic arterial blood pressure and SAP corresponds to the systolic arterial blood pressure of the patient.

In such a case, in method step (c) of the first iteration loop, the following formula might be applied for calculating the approximated pulse wave pulseₐₚₚᵣₒₓ(t);

$${pulse}_{approx} ( t ) = \frac{1}{N} {\sum }_{n = 1}^{N} {pulse}_{n \_ weighted} ( t ) \times {weight 1}_{n} .$$

As mentioned above, the weighting function applied in the second and/or higher iteration loop is preferably a differential pressure function having the clamp pressure clampₙ(t) as an input parameter and having the approximated pulse wave pulseₐₚₚᵣₒₓ(t) obtained in the previous iteration loop as another input parameter.

For example, the weighting function applied in the second and/or higher iteration loop can be a triangular function, preferably having its maximum when the clamp pressure clampₙ(t) equals a predetermined difference, preferably zero, to the approximated pulse wave pulseₐₚₚᵣₒₓ(t) obtained in a previous iteration loop.

If a triangular function is applied as weighting function weightₙ(t) in the second and/or higher iteration loop, the weighting function weightₙ(t) might be calculated as follows:

$$\begin{matrix}{weight}_{n} ( t ) = 1 - \frac{{clamp}_{n} - {pulse}_{approx \_ prev} ( t )}{{clamp}_{n} - {clamp}_{n - 1}} & if\end{matrix} {clamp}_{n - 1} < {pulse}_{approx \_ prev} ( t ) < {clamp}_{n} ;$$

$${wei ght}_{n} ( t ) = 1 - \frac{{pulse}_{approx \_ prev} ( t ) - {clamp}_{n}}{{clamp}_{n + 1} - {clamp}_{n}} if {clamp}_{n} \leq {pulse}_{approx \_ prev} < {clamp}_{n + 1} ;$$

$$and$$

$${weight}_{n} ( t ) = 0 otherwise .$$

As mentioned above, the index n refers to the number of the heart beat of the corresponding measured pulse signal pulse_{n_measured}(t). As mentioned above, even though, the clamp pressure clampₙ may not be constant over the detection period of one pulse wave, for the sake of simplicity, the clamp pressure clampₙ might be considered as being substantially constant during this detection period, e.g. corresponding to the clamp pressure clampₙ=clampₙ(t=t_{beat_n},) at the beginning of the corresponding detection period. pulse_{approx_prev}(t) corresponds to the result, i.e. the approximated pulse wave of the patient, of the previous iteration loop.

As an alternative to a triangular weighting function, the weighting function applied in the second and/or higher iteration loop may be a bell-shaped function, preferably having its maximum when the clamp pressure clampₙ(t) equals a predetermined difference, preferably zero, to the approximated pulse wave pulseₐₚₚᵣₒₓ(t) obtained in a previous iteration loop.

When using a bell-shaped function as weighting function weight_{approx_n}(t) in the second and/or higher iteration loop, the weighting function weightₙ(t) might be calculated as follows:

$${weight}_{n} ( t ) = \frac{1}{1 + {( \frac{{clamp}_{n} - {pulse}_{approx \_ prev} ( t )}{{p}_{w}} )}^{2}}$$

wherein parameter p_{w} corresponds to an empirically determined parameter being decisive for the width at half maximum of the bell-shaped weighting function. The parameter p_{w} is preferably chosen in accordance with the particular circumstances of the blood pressure measurement that have an influence on the distortion of the measured pulse curves. If distortion of the measured pulse curves increases (e.g. due to the use of another blood pressure measurement device), the increase or decrease rate of the cuff pressure should be decreased so as to measure more pulses of the patient within the detection time. In such a case, a smaller value for the parameter p_{w} may be chosen. Generally, the parameter p_{w} may preferably be chosen according to the following equation:

$${p}_{w} = \frac{SAP - DAP}{N} ,$$

wherein N is the total number of pulses measured during the detection period, i.e. measured substantially during the time needed by the cuff pressure to change from the diastolic arterial blood pressure DAP to the systolic arterial blood pressure SAP of the patient, or the other way around.

Preferably, method step (c) of the second and/or higher iteration loop further comprises: scaling the approximated pulse wave pulseₐₚₚᵣₒₓ(t) to the difference between the diastolic blood pressure value DAP and the systolic blood pressure value SAP of the patient. An example of such a scaling is provided below.

Scaling the approximated pulse wave pulseₐₚₚᵣₒₓ(t) ensures that the amplitude of the (scaled) approximated pulse wave correctly corresponds to the amplitude of the actual pulse wave of the patient. That is, the approximated pulse wave pulseₐₚₚᵣₒₓ(t) is scaled in such a way that its lower extreme value substantially corresponds to the diastolic blood pressure value DAP of the patient, whereas its upper extreme value substantially corresponds to the systolic blood pressure value SAP of the patient. As mentioned before, it is well-known to those skilled in the art, how to determine diastolic and systolic arterial blood pressure values DAP and SAP based on the so-called “oscillometric non-invasive blood pressure measurement method”.

If the approximated pulse wave pulseₐₚₚᵣₒₓ(t) is scaled in method step (c) of the second and/or higher iteration loop, the scaled approximated pulse wave pulse_{approx_scaled}(t) (instead of the approximated pulse wave pulseₐₚₚᵣₒₓ(t)) is applied in method step (b) of the subsequent iteration loop.

Similarly, method step (a) may further comprise: scaling the measured pulse signals pulse_{n_measured}(t) to the difference between the diastolic blood pressure value DAP and the systolic blood pressure value SAP of the patient.

Scaling of the measured pulse signals pulse_{n_measured}(t) in method step (a) might be performed by applying the following formula:

pulse_{n_measured_scaled}(t)=offsetₙ+scaleₙ×pulse_{n_measured}(t),

wherein the parameter offsetₙ is preferably calculated as follows:

offsetₙ=DAP−min(pulse_{n_measured}(t)), and

wherein the parameter scaleₙ is preferably calculated as follows:

$${scale}_{n} = \frac{SAP - DAP}{max ( {pulse}_{n \_ measured} ( t ) ) - min ( {pulse}_{n \_ measured} ( t ) )} .$$

max(pulse_{n_measured}(t)) corresponds the maximum value of the measured pulse wave corresponding to the heart beat with the number n. Similarly, min(pulse_{n_measured}(t)) corresponds the minimum value of the measured pulse wave corresponding to the heart beat with the number n.

Of course, as will be apparent to those skilled in the art, other formulas may be applied to calculate the parameters offsetₙ and scaleₙ. For example the formulas might equally be based on the mean arterial pressure MAP of the patient, which can also be determined based on the so-called “oscillometric non-invasive blood pressure measurement method”.

If the measured pulse signals pulse_{n_measured}(t) are scaled in method step (a), the scaled measured pulse signals pulse_{n_measured_scaled}(t) (instead of the measured pulse signals pulse_{n_measured}(t)) are applied in method step (b) to determine the weighted pulse signals pulse_{n_weighted}(t).

In such a case, in method step (c) of the first iteration loop, the following formula might be applied for calculating the approximated pulse wave pulseₐₚₚᵣₒₓ(t):

$${pulse}_{approx} ( t ) = \frac{1}{{\sum }_{n = 1}^{N} {weight}_{n} ( t )} {\sum }_{n = 1}^{N} {pulse}_{n \_ measured \_ scaled} ( t ) \times {weight}_{n} ( t ) ,$$

wherein the weighting function weightₙ(t) applied in the first iteration loop is preferably a function of a difference between scaled measured pulse signals pulse_{n_measured_scaled}(t) and the clamp pressure clampₙ(t).

According to another aspect, the invention refers to a logic unit for approximating a patient's pulse wave based on a non-invasive blood pressure measurement, configured to carry out the following steps:
weighting previously measured pulse signals pulse_{n_measured}(t) using a weighting function to obtain weighted pulse signals pulse_{n_weighted}(t);
adding up the weighted pulse signals pulse_{n_weighted}(t) to obtain an approximation of the patient's pulse wave pulseₐₚₚᵣₒₓ(t),
   wherein these steps are preferably iteratively repeated at least one more time.

The logic unit according to the present invention is configured to carry out the above described method, wherein the sequence of pulse signals pulse_{n_measured}(t) has been previously measured and stored, so that method step (a) can be skipped and the logic unit according to the present invention directly starts with method step (b), based on the previously stored signal values.

According to yet another aspect, the present invention also refers to a system for approximating a patient's pulse wave based on a non-invasive blood pressure measurement, comprising the logic unit described above and a blood pressure measurement device, the blood pressure measurement device being configured for non-invasively measuring a sequence n=1 . . . N of pulse signals of a patient to obtain measured pulse signals pulse_{n_measured}(t), wherein the system is configured for providing the measured pulse signals pulse_{n_measured}(t) as input values to the logic unit. Thus, the system is also configured to obtain the measured pulse signals pulse_{n_measured}(t) according to step (a) of the above described method.

Preferably, the blood pressure measurement device comprises a pressure cuff, and even more preferably, the pressure cuff is configured for being disposed around a patient's arm so as to measure the patient's arterial blood pressure in a non-invasive way. Thus, the system is configured to obtain the measured pulse signals pulse_{n_measured}(t) using the above-described “oscillometric non-invasive blood pressure measurement method”. Since the pressure cuff is configured for being attached around a patient's arm, preferably an upper arm of the patient, substantially no interference effects caused by pressure reflections adversely affect the measurement—contrary to the above described “volume-clamp-method”.

Even though, the “oscillometric non-invasive blood pressure measurement method” exhibits the advantage that substantially no interference effects caused by pressure reflections adversely affect the measurement (in contrast to known methods for measuring peripheral blood pressure waveform data, such as the above described “volume-clamp-method” utilized on a finger or the so called “applanation-tonometry-method” utilized at the patient's wrist), the “oscillometric non-invasive blood pressure measurement method” does not allow for continuous measurements without blocking blood flow in an unallowable manner. However, continuous measurement can be performed with the “volume-clamp-method” or with the “applanation-tonometry-method”.

As described above, it has already been tried in the past to overcome the disadvantages of the known methods for measuring peripheral blood pressure waveform data, such as the “volume-clamp-method” and the “applanation-tonometry-method”, by using transfer functions in order to reconstruct the central blood pressure waveforms from peripherally measured signals, e.g. signals measured at a patient's finger. However, since the applied transfer functions are usually based on statistical and empirical parameters that are not (or at least not sufficiently) adapted to the individual patient and situation, such an approach bears the likelihood to provide imprecise results.

Using pulse signals measured e.g. with the above described “oscillometric non-invasive blood pressure measurement method” to calibrate the transfer function to an individual patient did not represent a promising approach, either, since, in the past, it was not possible to approximate (with sufficient quality) the patient's pulse wave based on the measured pulse signals. However, with the method according to the present invention, an approximation of good quality of the patient's pulse wave based on non-invasive blood pressure measurement becomes possible. Therefore, the system described above may be combined with a device for measuring peripheral blood pressure waveform data, and a transfer function may be applied, wherein the transfer function is calibrated to an individual patient based on the patient's pulse wave that has been previously approximated according to the method of the present invention. That way, the central arterial blood pressure waveforms can be continuously determined with high quality.

Thus, the previously described system for approximating a patient's pulse wave based on a non-invasive blood pressure measurement preferably further comprises a second blood pressure measurement device that is adapted for non-invasively measuring peripheral blood pressure waveform data of the patient in a continuous way, wherein the system is adapted to apply a transfer function to reconstruct central blood pressure waveforms from the measured peripheral blood pressure waveform data based on the approximated pulse wave pulseₐₚₚᵣₒₓ(t).

The patient's pulse wave may be approximated according to the inventive method only once, preferably just before the continuous measurement of the peripheral blood pressure waveform data.

More preferably, the approximated pulse wave pulseₐₚₚᵣₒₓ(t) of the patient is yet determined at substantially regular intervals, wherein the transfer function is regularly recalibrated based on the regularly determined approximated pulse wave pulseₐₚₚᵣₒₓ(t) of the patient. For example, the pulse wave may be approximated according to the method of the present invention every two minutes. This way, it is possible to continuously obtain central blood pressure waveforms of very good quality.

For example, applying the transfer function may comprise the following steps: In a first step, both time-varying signals, i.e. the intermittent determined approximated pulse waves pulseₐₚₚᵣₒₓ(t) and the continuously measured peripheral blood pressure waveforms, are transformed into the frequency domain. Then, in a second step, the transfer function is determined. In a third step, the transfer function is applied to the peripherally measured blood pressure waveforms so as to calibrate the peripheral blood pressure waveforms. Finally, in a fourth step, the calibrated peripheral blood pressure waveforms are re-transformed into the time domain.

Exemplary embodiments of the present invention are described in more detail below based on the figures, in which:

FIG. 1: schematically shows a known pressure cuff configuration used to carry out the so-called “oscillometric non-invasive blood pressure measurement method”;

FIG. 2: schematically illustrates an electrocardiogram signal (ECG-signal) over the time, which signal has been measured with the pressure cuff configuration shown in FIG. 1;

FIG. 3: schematically illustrates the signal of the manometer of the pressure cuff configuration shown in FIG. 1 over the time;

FIG. 4: represents an exemplary diagram showing exclusively the pulses, i.e.

pressure oscillations caused by the patient's heart beats, indicated by the manometer over the time, thereby omitting the pressure variation caused by the continuously increasing cuff pressure;

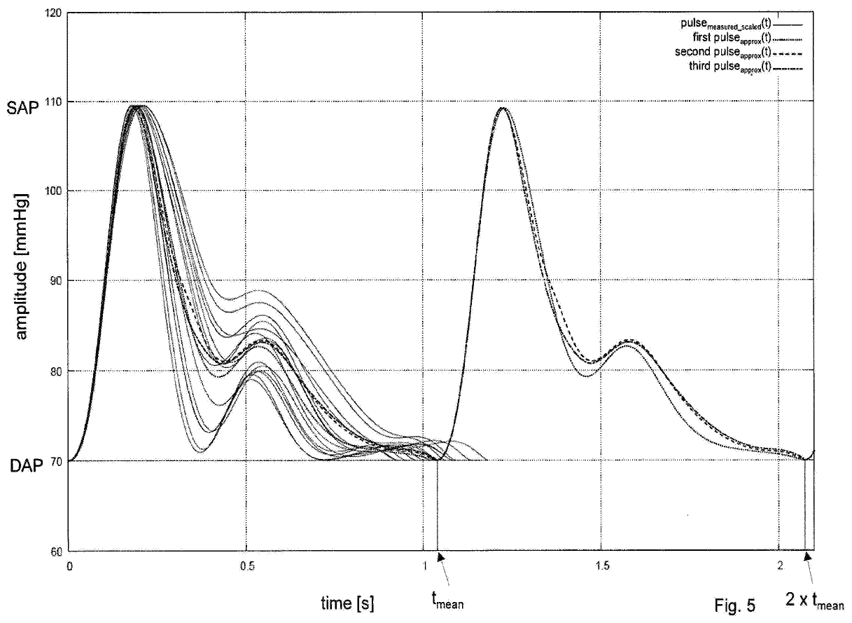

FIG. 5: illustrates an exemplary diagram showing approximated pulse waves of the patient, determined according to the method of the present invention;

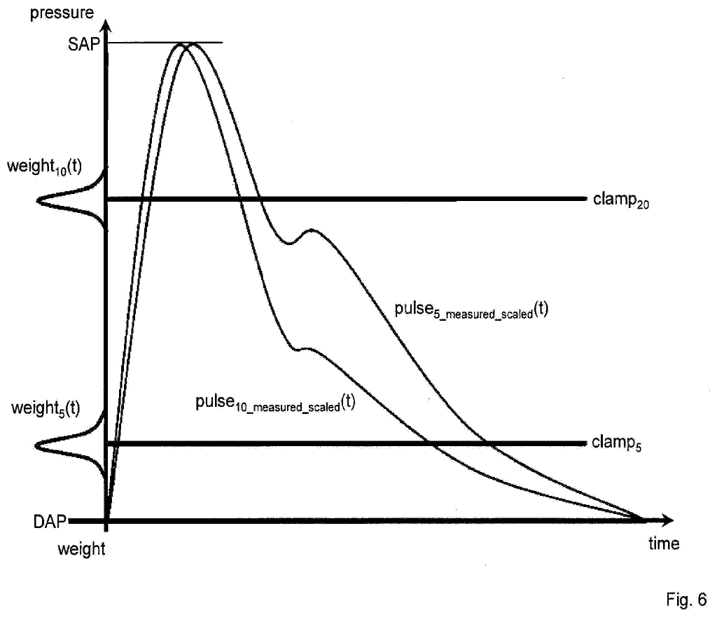

FIG. 6: illustrates the functioning of a first iteration loop of the method of the present invention;

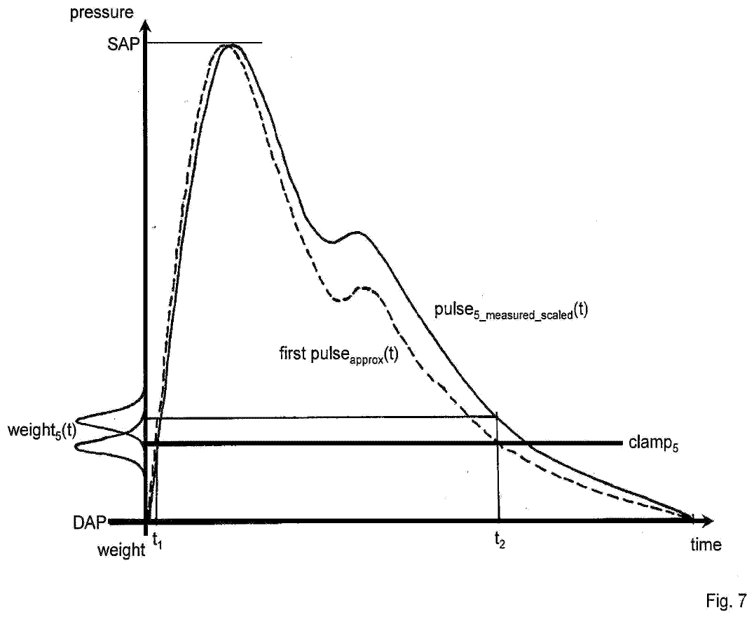

FIG. 7: illustrates the functioning of a second and/or higher iteration loop of the method of the present invention;

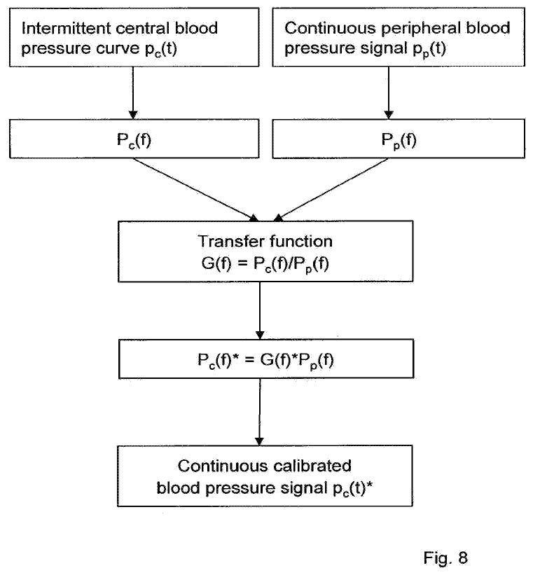

FIG. 8: illustrates a block diagram for continuously obtaining central blood pressure waveforms of high good quality.

As described above, FIGS. 1-4 all refer to a well-known method of non-invasively measuring blood pressure signals and to a conventional way of processing and representing the measured signals. In particular, FIG. 1 shows a known pressure cuff configuration comprising a manometer and used to carry out the so-called “oscillometric non-invasive blood pressure measurement method”. Furthermore, FIG. 4 is an exemplary diagram showing exclusively the pulses, i.e. pressure oscillations caused by the patient's heart beats, indicated by the manometer over the time, whereas the pressure variation caused by the continuously increasing cuff pressure is omitted in this diagram. As shown in FIG. 4, a sequence of pulse signals (caused by a corresponding number of heart beats of the patient) has been measured. The pressure oscillations shown in FIG. 4 are plotted in such a way that the curve cyclically oscillates around a zero pressure line (or zero pressure value). How to determine such a diagram is well known to those skilled in the art.

In the following, the present invention will be described in more detail in view of FIGS. 5-8.

FIG. 5 shows a diagram with time (in seconds) on its axis of abscissas and with pressure (in mmHg) on its axis of ordinates. Furthermore, a plurality (13 in this example, i.e. N=13 and n=1 . . . 13) of measured and scaled pulse signals pulse_{n_measured_scaled}(t) of a patient are represented in the diagram. In this example, the axis of abscissas corresponds to t-t_{beat_n} (as previously mentioned t_{beat_n} corresponds to the moment of beginning of a heart beat) so that the illustrated 13 curves pulse_{n_measured_scaled}(t) all start at 0 seconds with the same value, namely with the diastolic blood pressure value DAP of the patient.

The measured pulse signals pulse_{n_measured}(t) of a patient are scaled so as to make the amplitudes of the (scaled) measured pulse signals all correspond to the amplitude of the actual pulse wave of the patient. That is, the measured pulse signals pulse_{n_measured}(t) are scaled in such a way that the lower extreme value of each measured pulse signal substantially corresponds to the diastolic blood pressure value DAP of the patient, whereas its upper extreme value substantially corresponds to the systolic blood pressure value SAP of the patient. As mentioned before, it is well-known to those skilled in the art, how to determine diastolic and systolic arterial blood pressure values DAP and SAP, e.g. by applying the “oscillometric non-invasive blood pressure measurement method”.

Even though the measured and scaled pulse signals pulse_{n_measured_scaled}(t) all have the same amplitude, FIG. 5 clearly shows that they significantly differ from each other with respect to their wave form.

Furthermore, a curve named “first pulseₐₚₚᵣₒₓ(t)” is illustrated in FIG. 5 by a dotted line. This curve has been determined in the first iteration loop of the method according to the invention by simply averaging the curves of the measured and scaled pulse signals pulse_{n_measured_scaled}(t). Notably, only those measured pulse signals pulse_{n_measured}(t) have been taken into account that have been measured with the clamp pressure clampₙ(t) being between the diastolic and the systolic pressure DAP and SAP of the patient.

In other words, the following formula has been applied for calculating the approximated pulse wave first pulseₐₚₚᵣₒₓ(t) of the first iteration loop:

$$first\_puls {e}_{approx} ( t ) = \frac{1}{N} {\sum }_{n = 1}^{N} {pulse}_{n \_ weighted \_ scaled} ( t ) \times weight {1}_{n} , with \text{:}$$

$$weight {1}_{n} = 1 if DAP < {clamp}_{n} ( t ) < S A P , \\ {weight 1}_{n} = 0 otherwise .$$

However, instead of obtaining the approximated pulse wave first pulseₐₚₚᵣₒₓ(t) of the first iteration loop by simply averaging the curves of the measured and scaled pulse signals pulse_{n_measured_scaled}(t), the measured and scaled pulse signals pulse_{n_measured_scaled}(t) may be weighted in a more sophisticated way before adding them up. For example, a bell-shaped weighting function may be applied is schematically illustrated in FIG. 6.

FIG. 6 shows a schematic example of only two (for the sake of clarity) measured and scaled pulse signals pulse_{n_measured_scaled}(t), namely for the fifth (n=5) and the tenth (n=10) heart beat. A bell-shaped function is applied as weighting function weightₙ(t), so as to particularly accentuate those portions of the scaled measured and scaled pulse signals pulse_{n_measured_scaled}(t) that substantially correspond to the clamp pressure associated to the corresponding heart beat (clampₙ=clamp(t_{beat_n})).

Calculation of the bell-shaped weighting function weightₙ(t) for the first iteration loop works substantially analogue to the calculation of the bell-shaped weighting function weightₙ(t) for the second or higher iteration loop, which has been described in detail above.

Generally, the approximated pulse wave pulseₐₚₚᵣₒₓ(t) lasts on the axis of the abscissas t-t_{beat_n} from 0 seconds till a mean pulse duration time tₘₑₐₙ of the measured and scaled pulse signals pulse_{n_measured_scaled}(t). In the example shown in FIG. 5, the mean pulse duration time tₘₑₐₙ is at about 1.05 seconds. For determining the approximated pulse wave pulseₐₚₚᵣₒₓ(t), if the duration of a corresponding pulse of the measured and scaled pulse signals pulse_{n_measured_scaled}(t) is shorter than the mean pulse duration time tₘₑₐₙ, the latest samples of the measured and scaled pulse signals pulse_{n_measured_scaled}(t) of the corresponding pulse are duplicated. On the other hand, if duration of a corresponding pulse of the measured and scaled pulse signals pulse_{n_measured_scaled}(t) is longer than the mean pulse duration time tₘₑₐₙ, the samples after the mean pulse duration time tₘₑₐₙ are simply omitted.

Moreover, FIG. 5 also exhibits a curve named “second pulseₐₚₚᵣₒₓ(t)” illustrated by a dashed line. This curve represents an approximation of the patient's pulse wave obtained in a second iteration loop of the method according to the invention, by applying a bell-shaped weighting function, as will be explained in more detail in view of FIG. 7.

FIG. 7 schematically shows only one (for the sake of clarity) of the measured and scaled pulse signals pulse_{n_measured_scaled}(t), namely for the fifth (n=5) heart beat. Additionally, FIG. 5 shows—indicated by a dashed line—the approximated pulse wave first pulseₐₚₚᵣₒₓ(t) determined in the first iteration loop. As an approximation, it is assumed that the approximated pulse wave first pulseₐₚₚᵣₒₓ(t) determined in the first iteration loop corresponds to the actual pulse wave of the patient. In the present example, the approximated pulse wave first pulseₐₚₚᵣₒₓ(t) equals the clamp pressure clamp₅=clamp(t_{beat_5}) two times, namely at moments t₁ and t₂, as shown in FIG. 7. At these moments, the fifth measured and scaled pulse signals pulse_{5_measured_scaled}(t) is assumed to exhibit a linear relationship with respect to the actual internal arterial blood pressure. Therefore, the fifth measured and scaled pulse signal pulse_{5_measured_scaled}(t) is weighted so as to particularly accentuate portions of that curve corresponding to moments t₁ and t₂. As further shown in FIG. 7, a bell-shaped function is applied as weighting function weight₅(t) for weighting the fifth measured and scaled pulse signal curve pulse_{5_measured_scaled}(t). As can be seen from FIG. 7, the value of the fifth measured and scaled pulse signal curve pulse_{5_measured_scaled}(t) at moment t₁ is substantially equal to the value of the weighting function weight₅(t) at moment t₁. However, the value of the fifth measured and scaled pulse signal curve pulse_{5_measured_scaled}(t) at moment t₂ significantly differs from the value of the weighting function weight₅(t) at moment t₂.

The same is repeated for all measured and scaled pulse signals pulse_{n_measured_scaled}(t), wherein always those portions of the signal curves are particularly accentuated which correspond to moments at which the approximated pulse wave first pulseₐₚₚᵣₒₓ(t) determined in the first iteration loop substantially equals the corresponding clamp pressure clampₙ. Then, the weighted pulse signals pulse_{n_weighted}(t) are added up to obtain a better approximation of the patient's pulse wave second pulseₐₚₚᵣₒₓ(t) as result of the second iteration loop.

Notably, it is not necessary to accentuate the portions of the signal curves which correspond to moments at which the approximated pulse wave first pulseₐₚₚᵣₒₓ(t) determined in the first iteration loop substantially equals the corresponding clamp pressure clampₙ (i.e. the difference is zero). Instead, it is also possible to apply another difference, as long as the same difference is applied for weighting all of the measured and scaled pulse signals pulse_{n_measured_scaled}(t).

Finally, FIG. 5 also exhibits a curve named “third pulseₐₚₚᵣₒₓ(t)” illustrated by a chain dotted line. This curve is obtained as a result of the third iteration loop of the method of the present invention in a substantially analogous way as the curve named “second pulseₐₚₚᵣₒₓ(t)”. However, instead of applying the curve named “first pulseₐₚₚᵣₒₓ(t)” as an approximation of the actual pulse wave of the patient, in the third iteration loop, the curve named “second pulseₐₚₚᵣₒₓ(t)” is applied. As can be seen from FIG. 5, the result of the third iteration loop is already quite similar to the result of the second iteration loop. However, if desired, further iteration loops may be carried out.

It should be noted that in FIG. 5, only for the sake of clarity, the curve “first pulseₐₚₚᵣₒₓ(t)”, the curve “second pulseₐₚₚᵣₒₓ(t)”, and the curve “third pulseₐₚₚᵣₒₓ(t)”are each shown two times in FIG. 5, one time lasting on the axis of the abscissas t-t_{beat_n} from 0 seconds till tₘₑₐₙ, and (additionally) a second time lasting from tₘₑₐₙ till 2×tₘₑₐₙ.

FIG. 8 illustrates a block diagram for a method of continuously obtaining central blood pressure waveforms of high quality. This method can be carried out by a system according to the present invention, the system comprising a logic unit capable of performing the method according to the present invention and a first blood pressure measurement device being configured for non-invasively measuring a sequence n=1 . . . N of pulse signals of a patient to obtain measured pulse signals pulse_{n_measured}(t) as input values for the logic unit. Preferably, the first blood pressure measurement device comprises a pressure cuff adapted for being disposed around a patient's upper arm to measure the patient's arterial blood pressure in a non-invasive way, as shown e.g. in FIG. 1. The system further comprises a second blood pressure measurement device that is adapted for non-invasively measuring peripheral blood pressure waveform data of the patient in a continuous way. The second blood pressure measurement device is preferably capable to perform the volume-clamp-method, described above.

With such a system, the method shown in FIG. 8 can be carried out. According to this method, the approximated pulse wave pulseₐₚₚᵣₒₓ(t) of the patient is repeatedly determined according to the method of the present invention, preferably at substantially regular intervals, using the first blood pressure measurement device, thereby obtaining intermittent central arterial blood pressure curves p_{c}(t). At the same time, peripheral blood pressure signals pₚ(t) are continuously measured by the second blood pressure measurement device.

E.g. the intermittent central blood pressure curve p_{c}(t) and the peripheral blood pressure signal pₚ(t) are then both transformed into the frequency domain, so as to obtain a central blood pressure signal curve in the frequency domain P_{c}(f) and a peripheral blood pressure signal curve in the frequency domain Pₚ(f).

Next, a transfer function G(f) is calculated, based on the central blood pressure signal curve in the frequency domain P_{c}(f) and a peripheral blood pressure signal curve in the frequency domain Pₚ(f).

A calibrated blood pressure signal curve P_{c}(f)* can then be simply obtained by multiplying the transfer function G(f) with the a peripheral blood pressure signal curve in the frequency domain Pₚ(f).

Finally, a calibrated blood pressure curve signal p_{c}(t)* is determined by transforming the calibrated blood pressure signal curve P_{c}(f)* again into the time domain.

1-17. (canceled)

18. A non-invasive blood pressure measurement system comprising:
a pressure cuff,
wherein the pressure cuff is applied over a blood vessel,
wherein the pressure cuff is configured to receive one or more non-constant clamp pressure signals,
wherein the one or more non-constant clamp pressure signals cause the pressure cuff to at least one of inflate, deflate, and maintain pressure; and
one or more computer processors coupled to one or more computer readable storage devices;
program instructions to receive a pulse curve signal from the pressure cuff, wherein the pulse curve signal represents a sequence of pulse curves of a subject;
program instructions to apply a weighting function,
wherein the weighting function applies one or more weights to a pulse curve of the sequence of pulse curves to obtain a weighted pulse curve, and
wherein the one or more non-constant clamp pressure signals are a weighting function input parameter;
program instructions to sum at least the weighted pulse curve and a second weighted pulse curve, wherein the sum approximates a shape and an amplitude of a pulse wave of the subject;
program instructions to determine a health status of the subject based on the pulse wave of the subject;
program instructions to generate a graphical representation of the pulse wave of the subject and a health status indicator.

19. The system of claim 18, further comprising program instructions to send a display signal to a patient monitor, wherein the display signal causes the patient monitor to display the graphical representation of the pulse wave and the health status indicator.

20. The system of claim 18, wherein the shape and the amplitude of the pulse wave of the subject is displayed as a curve expressed as pressure versus time of a blood pressure oscillation representing a heartbeat of the subject.

21. The system of claim 18, wherein a clamp pressure of the pressure cuff is at least one of increased and decreased between a diastolic blood pressure value and a systolic blood pressure value of the subject.

22. The system of claim 18, further comprising;
program instructions to apply a revised weighting function, wherein the revised weighting function applies one or more revised weights to a second pulse curve of a second sequence of pulse curves to obtain a third weighted pulse curve; and
program instructions to sum at least the third weighted pulse curve and a fourth weighted pulse curve, wherein the sum approximates a shape and an amplitude of an updated pulse wave of the subject.

23. The system of claim 22, wherein the shape and the amplitude of the pulse wave is a revised weighting function input parameter.

24. The system of claim 22, wherein the revised weighting function is a bell-shaped function, and wherein a maximum of the bell-shaped function occurs when the non-constant clamp pressure equals a predetermined difference relative to the pulse wave obtained using the weighting function.

25. A non-invasive blood pressure measurement device, comprising:
a display;
one or more computer processors coupled to one or more computer readable storage devices;
program instructions to receive a pulse curve signal from a pressure cuff, wherein the pulse curve signal represents a sequence of pulse curves of a subject;
program instructions to apply a weighting function,
wherein the weighting function applies one or more weights to a pulse curve of the sequence of pulse curves to obtain a weighted pulse curve, and
wherein the one or more non-constant clamp pressure signals are a weighting function input parameter;
program instructions to sum at least the weighted pulse curve and a second weighted pulse curve, wherein the sum approximates a shape and an amplitude of a pulse wave of the subject;
program instructions to determine a health status of the subject based on the pulse wave of the subject;
program instructions to generate a graphical representation of the pulse wave of the subject and the health status of the subject.

26. The device of claim 25, further comprising program instructions to display the graphical representation of the pulse wave and the health status of the subject.

27. The device of claim 25, wherein the shape and the amplitude of the pulse wave of the subject is displayed as a curve expressed as pressure versus time of a blood pressure oscillation representing a heartbeat of the subject.

28. The system of claim 25, wherein a clamp pressure of the pressure cuff is at least one of increased and decreased between a diastolic blood pressure value and a systolic blood pressure value of the subject.

29. The device of claim 25, further comprising;
program instructions to apply a revised weighting function, wherein the revised weighting function applies one or more revised weights to a second pulse curve of a second sequence of pulse curves to obtain a third weighted pulse curve; and
program instructions to sum at least the third weighted pulse curve and a fourth weighted pulse curve, wherein the sum approximates a shape and an amplitude of an updated pulse wave of the subject.

30. The system of claim 29, wherein the shape and the amplitude of the pulse wave is a revised weighting function input parameter.

31. The system of claim 29, wherein the revised weighting function is a bell-shaped function, and wherein a maximum of the bell-shaped function occurs when the non-constant clamp pressure equals a predetermined difference relative to the pulse wave obtained using the weighting function.

32. A method of non-invasively measuring blood pressure, comprising:
receiving, by one or more computer processors, a pulse curve signal representing a sequence of pulse curves associated with a subject;
applying, by the one or more computer processors, a weighting function,
wherein the weighting function applies one or more weights to a pulse curve of the sequence of pulse curves to obtain a weighted pulse curve, and
wherein the one or more non-constant clamp pressure signals are a weighting function input parameter;
summing, by the one or more computer processors, at least the weighted pulse curve and a second weighted pulse curve, wherein the sum approximates a shape and an amplitude of a pulse wave of the subject,
determining, by the one or more computer processors, a health status of the subject based on the pulse wave of the subject;
generating, by the one or more computer processors, a graphical representation of the pulse wave of the subject and a health status indicator.

33. The method of claim 32, further comprising sending, by the one or more computer processors, a display signal to a patient monitor, wherein the display signal causes the patient monitor to display the graphical representation of the pulse wave and the health status indicator.

34. The device of claim 32, wherein the shape and the amplitude of the pulse wave of the subject is displayed as a curve expressed as pressure versus time of a blood pressure oscillation representing a heartbeat of the subject.

35. The method of claim 32, further comprising;
applying, by the one or more computer processors, a revised weighting function, wherein the revised weighting function applies one or more revised weights to a second pulse curve of a second sequence of pulse curves to obtain a third weighted pulse curve; and
summing, by the one or more computer processors, at least the third weighted pulse curve and a fourth weighted pulse curve, wherein the sum approximates a shape and an amplitude of an updated pulse wave of the subject.

36. The system of claim 35, wherein the shape and the amplitude of the pulse wave is a revised weighting function input parameter.

37. The system of claim 35, wherein the revised weighting function is a bell-shaped function, and wherein a maximum of the bell-shaped function occurs when the non-constant clamp pressure equals a predetermined difference relative to the pulse wave obtained using the weighting function.
